(19) United States
(12) Patent Application Publication
(10) Pub. No.: US 2024/0246891 A1
(43) Pub. Date: Jul. 25, 2024

(54) METHOD FOR PRODUCING TRIFLUOROETHYLENE AND RECYCLING THE CHLOROTRIFLUOROETHYLENE STREAM

(71) Applicant: ARKEMA FRANCE, COLOMBES (FR)

(72) Inventors: Alexandre CAMBRODON, Pierre-Benite Cedex (FR); Cédric LAVY, Pierre-Benite Cedex (FR); Thierry LANNUZEL, Pierre-Benite Cedex (FR); Philippe LEDUC, Pierre-Benite Cedex (FR); Kevin HISLER, Pierre-Benite Cedex (FR)

(21) Appl. No.: 18/564,332

(22) PCT Filed: Jun. 3, 2022
(86) PCT No.: PCT/FR2022/051054

§ 371 Date: Nov. 27, 2023

(30) Foreign Application Priority Data
Jun. 7, 2021 (FR) FR2105962

(51) Int. Cl. C07C 17/25; B01D 3/36; B01J 21/04; B01J 23/44; C07C 17/386

(52) U.S. Cl. CPC C07C 17/25; B01D 3/36; B01J 21/04; B01J 23/44; C07C 17/386

(57) ABSTRACT

The present invention relates to a process for producing trifluoroethylene in a reactor provided with a fixed catalytic bed comprising a catalyst, said process comprising the steps of: a) reacting chlorotrifluoroethylene with hydrogen in the presence of the catalyst and in the gas phase to produce a stream A comprising trifluoroethylene; b) treating said stream A under conditions sufficient to produce a stream D1 comprising 1,1,2-trifluoroethane in a content of less than 15% by weight on the basis of the total weight of said stream D1, c) recycling stream D1 to step a).

### TECHNICAL FIELD

The present invention relates to a process for the production of hydrofluoroolefins. In particular, the present invention relates to a process for the production of trifluoroethylene (VF₃) by hydrogenolysis of chlorotrifluoroethylene.

### TECHNOLOGICAL BACKGROUND OF THE INVENTION

Fluorinated olefins, such as VF₃, are known and are used as monomers or comonomers for the manufacture of fluorocarbon polymers exhibiting noteworthy characteristics, in particular excellent chemical resistance and good heat resistance.

Trifluoroethylene is a gas under standard conditions of pressure and temperature. The main risks associated with the use of this product relate to its flammability, its propensity for self-polymerization when it is not stabilized, its explosiveness due to its chemical instability and its supposed sensitivity to peroxidation, by analogy with other halogenated olefins. Trifluoroethylene has the particularity of being extremely flammable, with a lower explosive limit (LEL) of about 10% and an upper explosive limit (UEL) of about 30%. The major danger, however, is associated with the propensity of VF₃ to decompose violently and explosively under certain pressure conditions in the presence of an energy source, even in the absence of oxygen. Given the main risks above, the synthesis and also the storage of VF₃ pose particular problems and impose strict safety rules throughout these processes. A known route for the preparation of trifluoroethylene uses, as starting materials, chlorotrifluoroethylene (CTFE) and hydrogen in the presence of a catalyst and in the gas phase. A process is known from WO2013/128102 for the production of trifluoroethylene by hydrogenolysis of CTFE in the gas phase and in the presence of a catalyst based on a metal from group VIII at atmospheric pressure and at relatively low temperatures.

### SUMMARY OF THE INVENTION

According to a first aspect, the present invention relates to a process for the production of trifluoroethylene in a reactor provided with a fixed catalytic bed comprising a catalyst, said process comprising the steps of:
a) reacting chlorotrifluoroethylene with hydrogen in the presence of the catalyst and in the gas phase in order to produce a stream A comprising trifluoroethylene;
b) treating said stream A under conditions sufficient to produce a stream D1 comprising 1,1,2-trifluoroethane in a content of less than 15% by weight on the basis of the total weight of said stream D1;
c) recycling stream D1 to step a).

According to a preferred embodiment, stream A also comprises, in addition to trifluoroethylene, unreacted chlorotrifluoroethylene and 1,1,2-trifluoroethane.

According to a preferred embodiment, stream D1 also comprises chlorotrifluoroethylene in a content by weight greater than 60% by weight on the basis of the total weight of said stream D1.

According to a preferred embodiment, stream D1 is in the form of an azeotropic or quasi-azeotropic composition comprising chlorotrifluoroethylene and 1,1,2-trifluoroethane.

The presence and formation of an azeotrope or quasi-azeotrope between chlorotrifluoroethylene and 1,1,2-trifluoroethane has been identified. The existence of an azeotrope complicates the process for purifying trifluoroethylene and in particular the process for recycling chlorotrifluoroethylene. However, it has been observed, surprisingly, that the recycling of a portion of the azeotrope is not detrimental to the production of trifluoroethylene. In fact, it has been observed that the yield of the hydrogenolysis reaction is not affected by a 1,1,2-trifluoroethane content of less than 15% in the recycle stream. Thus, it is not necessary to completely remove the 1,1,2-trifluoroethane from the recycle stream. This makes it possible to simplify the operations of purifying the recycle stream and therefore represents a significant economic gain.

According to a preferred embodiment, stream A comprises trifluoroethylene, chlorotrifluoroethylene and 1,1,2-trifluoroethane and step b) comprises the steps of:
b1) purifying said stream A comprising trifluoroethylene, chlorotrifluoroethylene and 1,1,2-trifluoroethane to form a stream C1 comprising trifluoroethylene and a stream C2 comprising chlorotrifluoroethylene and 1,1,2-trifluoroethane;
b2) purifying stream C2 to produce said stream D1 and a stream D2 comprising 1,1,2-trifluoroethane.

According to a preferred embodiment, step b2) is carried out by distillation at a pressure of less than 8 bara, preferably less than 6 bara.

According to a preferred embodiment, step b2) is carried out by distillation, and the temperature at the top of the distillation column is less than 40° C.

According to a preferred embodiment, said catalyst is a catalyst based on a metal from groups 8 to 10 of the Periodic Table of the Elements, preferably deposited on a support, in particular an aluminum-based support.

In a preferred embodiment, the catalyst comprises palladium supported on α-alumina.

According to a preferred embodiment, the chlorotrifluoroethylene and the hydrogen are in anhydrous form.

### DETAILED DESCRIPTION OF THE INVENTION

The present invention relates to a process for the production of trifluoroethylene comprising a step of hydrogenolysis reaction of chlorotrifluoroethylene (CTFE) with hydrogen in the gas phase and preferably in the presence of a catalyst.

According to a preferred embodiment, the process according to the invention described in the present application is carried out continuously.

According to a preferred embodiment, in the process described in the present application, the hydrogen is in anhydrous form.

According to a preferred embodiment, in the process described in the present application, the chlorotrifluoroethylene is in anhydrous form.

The implementation of the processes according to the invention in the presence of anhydrous hydrogen and/or chlorotrifluoroethylene makes it possible to effectively increase the lifetime of the catalyst and thus the overall productivity of the process. The term anhydrous refers to a content by weight of water of less than 1000 ppm, advantageously 500 ppm, preferably of less than 200 ppm, in particular of less than 100 ppm, on the basis of the total weight of the compound under consideration.

### Catalyst

Preferably, the catalyst is based on a metal from columns 8 to 10 of the Periodic Table of the Elements. In particular, the catalyst is based on a metal selected from the group consisting of Pd, Pt, Rh, and Ru; preferably palladium.

Preferably, the catalyst is supported. The support is preferably selected from the group consisting of activated carbon, an aluminum-based support, calcium carbonate, and graphite. Preferably, the support is based on aluminum. In particular, the support is alumina. The alumina may be α-alumina. Preferably, the alumina comprises at least 90% of α-alumina. It was observed that the conversion of the hydrogenolysis reaction was improved when the alumina is an α-alumina. Thus, the catalyst is more particularly palladium supported on alumina, advantageously palladium supported on an alumina comprising at least 90% of α-alumina, preferably palladium supported on an α-alumina.

Preferably, the palladium represents from 0.01% to 5% by weight on the basis of the total weight of the catalyst, preferably from 0.1% to 2% by weight, on the basis of the total weight of the catalyst.

In particular, said catalyst comprises from 0.01% to 5% by weight of palladium supported on alumina, the alumina preferably comprising at least 90% of α-alumina, more preferably the alumina being an α-alumina.

### Catalyst Activation

Said catalyst is preferably activated before its use in step a). Preferably, the activation of the catalyst is carried out at high temperature and in the presence of a reducing agent. According to a specific embodiment, the reducing agent is chosen from the group consisting of hydrogen, carbon monoxide, nitrogen monoxide, formaldehyde, C₁-C₆ alkanes and C₁-C₁₀ hydrohalocarbons, or a mixture of these; preferably hydrogen or a C₁-C₁₀ hydrohalocarbon, or a mixture of these; in particular hydrogen, chlorotrifluoroethylene, trifluoroethylene, chlorotrifluoroethane, trifluoroethane or difluoroethane, or a mixture of these. Preferably, the activation of the catalyst is carried out at a temperature of between 100° C. and 400° C., in particular at a temperature of between 150° C. and 350° C.

### Catalyst Regeneration

The catalyst used in the present process can be regenerated. This regeneration step can be carried out in a catalytic bed temperature range of between 90° C. and 450° C. Preferably, the regeneration step is carried out in the presence of hydrogen. Carrying out the regeneration step makes it possible to improve the reaction yield compared to the initial yield before regeneration. According to a preferred embodiment, the regeneration step can be carried out at a catalytic bed temperature of from 90° C. to 300° C., preferably at a catalytic bed temperature of from 90° C. to 250° C., more preferentially of from 90° C. to 200° C., in particular of from 90° C. to 175° C., more particularly at a catalytic bed temperature of from 90° C. to 150° C. In particular, the implementation of the regeneration step at a low temperature, for example from 90° C. to 200° C. or from 90° C. to 175° C. or from 90° C. to 150° C., makes it possible to desorb compounds which are harmful to the activity of the catalyst and/or to limit phase transitions which modify the structure of the catalyst.

According to another preferred embodiment, the regeneration step can be carried out at a catalytic bed temperature of greater than 200° C., advantageously greater than 230° C., preferably greater than 250° C., in particular greater than 300° C. The regeneration step may be carried out periodically as a function of the productivity or conversion obtained in step a). The regeneration step can be advantageously carried out at a catalytic bed temperature of between 200° C. and 300° C., preferably between 205° C. and 295° C., more preferentially between 210° C. and 290° C., in particular between 215° C. and 290° C., more particularly between 220° C. and 285° C., preferably between 225° C. and 280° C., more preferably between 230° C. and 280° C. Alternatively, the regeneration step may be carried out at a temperature of between 300° C. and 450° C., preferably between 300° C. and 400° C. The regenerated catalyst can be reused in step a) of the present process.

### Hydrogenolysis Reaction

The present invention comprises, as mentioned above, a step of a hydrogenolysis reaction of chlorotrifluoroethylene (CTFE) with hydrogen to produce a stream comprising trifluoroethylene. The hydrogenolysis step is carried out in the presence of a catalyst and in the gas phase. The hydrogenolysis step is preferably carried out in the presence of a pre-activated catalyst and in the gas phase. The hydrogenolysis step consists in simultaneously introducing hydrogen, CTFE and optionally an inert gas, such as nitrogen, in the gas phase and in the presence of said catalyst, preferably activated catalyst.

Preferably, said step a) is carried out at a temperature of the fixed catalytic bed of between 50° C. and 250° C. Said step a) can be carried out at a temperature of the fixed catalytic bed of between 50° C. and 240° C., advantageously between 50° C. and 230° C., preferably between 50° C. and 220° C., more preferentially between 50° C. and 210° C., in particular between 50° C. and 200° C. Said step a) can also be carried out at a temperature of the fixed catalytic bed of between 60° C. and 250° C., advantageously between 70° C. and 250° C., preferably between 80° C. and 250° C., more preferentially between 90° C. and 250° C., in particular between 100° C. and 250° C., more particularly between 120° C. and 250° C. Said step a) can also be carried out at a temperature of the fixed catalytic bed of between 60° C. and 240° C., advantageously between 70° C. and 230° C., preferably between 80° C. and 220° C., more preferentially between 90° C. and 210° C., in particular between 100° C. and 200° C., more particularly between 100° C. and 180° C., favorably between 100° C. and 160° C., particularly preferably between 120° C. and 160° C.

The H₂S/CTFE molar ratio is between 0.5/1 and 2/1, and preferably between 1/1 and 1.2/1. If an inert gas, such as nitrogen, is present in step a), the nitrogen/H₂ molar ratio is of between 0/1 and 2/1 and preferably of between 0/1 and 1/1.

Step a) is preferably carried out at a pressure of from 0.05 MPa to 1.1 MPa, more preferentially of from 0.05 MPa to 0.5 MPa, in particular at atmospheric pressure.

The contact time, calculated as being the ratio of the volume, in liters, of catalyst to the total flow rate of the gas mixture, in standard liters per second, at the inlet of the reactor, is of between 1 and 60 seconds, preferably between 5 and 45 seconds, in particular between 10 and 30 seconds, more particularly between 15 and 25 seconds.

### Reaction Stream Processing

The hydrogenolysis step (step a)) of the present process results in the production of a stream A comprising trifluoroethylene. Said stream A may also comprise unreacted hydrogen and unreacted chlorotrifluoroethylene. Stream A may also comprise 1,1,2-trifluoroethane as byproducts of the hydrogenolysis reaction. Stream A may also comprise HCl or HF or a mixture of both. Optionally, said stream A may also comprise 1,1-difluoroethane.

As mentioned above, step b) comprises the steps of:
b1) purifying said stream A comprising trifluoroethylene, chlorotrifluoroethylene and 1,1,2-trifluoroethane to form a stream C1 comprising trifluoroethylene and a stream C2 comprising chlorotrifluoroethylene and 1,1,2-trifluoroethane;
b2) purifying stream C2 to produce said stream D1 and a stream D2 comprising 1,1,2-trifluoroethane.

Said stream C1 comprising trifluoroethylene may also contain small amounts of chlorotrifluoroethylene and of 1,1,2-trifluoroethane. Preferably, the content by weight of chlorotrifluoroethylene in stream C1 is less than 10%, preferably less than 5%, in particular less than 1% by weight on the basis of the total weight of stream C1. Preferably, the content by weight of 1,1,2-trifluoroethane in stream C1 is less than 10%, preferably less than 5%, in particular less than 1% by weight on the basis of the total weight of stream C1.

Depending on the composition of said stream A, the purification thereof (step b1)) may comprise a plurality of steps. Thus, if said stream A comprises acid compounds such as HF or HCl, steps i) and ii) below can be carried out to remove them.

If said stream A comprises hydrogen and optionally inert gases, step iii) below can be carried out.

According to a particular embodiment, step b1) of the present process may comprise the steps of:
i) removing of HF and/or HCl from said stream A recovered in step a) to form a gas mixture B;
ii) drying the mixture B from step i);
iii) treating stream B dried in step ii) to remove hydrogen and optionally inert gases and to form a gas stream C;
iv) distilling said stream C to form a stream C1 comprising trifluoroethylene and a stream C2 comprising chlorotrifluoroethylene and 1,1,2-trifluoroethane.

The paragraph below details steps i) to iv).

Stream A is recovered at the reactor outlet in gaseous form. Preferably, at the outlet of the hydrogenolysis reactor, stream A is first of all treated in order to remove HCl and HF. Stream A is passed through water in a scrubbing column followed by scrubbing with a dilute base, such as NaOH or KOH. The remainder of the gas mixture, consisting of the unconverted reactants (H₂ and CTFE), dilution nitrogen (if present), trifluoroethylene, 1,1,2-trifluoroethane which form the gas mixture B, is directed to a dryer in order to remove the traces of scrubbing water. Drying can be carried out using products such as calcium, sodium or magnesium sulfate, calcium chloride, potassium carbonate, silica gel or zeolites. In one embodiment, a molecular sieve (zeolite), such as siliporite, is used for the drying. Stream B, thus dried, is subjected to a step of separation of the hydrogen and inert substances from the remainder of the other products present in the mixture B, by absorption/desorption in the presence of an alcohol comprising from 1 to 4 carbon atoms and preferably ethanol, at atmospheric pressure and at a temperature below ambient temperature, preferably of less than 10° C. and even more preferably at a temperature of −25° C., for the absorption. In one embodiment, the absorption of the organic substances is carried out in a countercurrent column with ethanol cooled to −25° C. The ethanol flow rate is adjusted according to the flow rate of organic substances to be absorbed. The hydrogen and inert gases, which are insoluble in ethanol at this temperature, are removed at the absorption column top. The organic substances are subsequently recovered in the form of a gas mixture C, by heating the ethanol to its boiling point (desorption), in order to be subsequently distilled. The mixture C is then purified, preferably distilled, to form a stream C1 comprising trifluoroethylene and a stream C2 comprising chlorotrifluoroethylene and 1,1,2-trifluoroethane. Stream C2 comprising chlorotrifluoroethylene and 1,1,2-trifluoroethane is recovered at the bottom of the column. Streams A, B, C and C2 may also contain 1,1-difluoroethane.

Purification of said mixture C2 (step b2)) makes it possible to produce said stream D1 and a stream D2 comprising 1,1,2-trifluoroethane. As mentioned above, said stream D1 comprises 1,1,2-trifluoroethane in a content by weight of less than 15% on the basis of the total weight of stream D1. Said stream D1 comprises, in addition to 1,1,2-trifluoroethane, chlorotrifluoroethylene and optionally 1,1-difluoroethane. Preferably, step b2) of the present process is carried out by distillation. Thus, stream D1 is recovered at the top of the distillation column. Stream D2 is, for its part, recovered at the bottom of the distillation column.

Step b2) is preferably carried out so as to obtain a stream D1 comprising chlorotrifluoroethylene and 1,1,2-trifluoroethane wherein the content by weight of 1,1,2-trifluoroethane is less than 15% on the basis of the total weight of said stream, preferably, wherein the content by weight of 1,1,2-trifluoroethane is from 0.01% to 15% on the basis of the total weight of said stream. More preferentially, said stream D1 comprises a content by weight of 1,1,2-trifluoroethane of less than 10% on the basis of the total weight of said stream D1, in particular from 0.01% to 10% of 1,1,2-trifluoroethane on the basis of the total weight of said stream D1.

According to a preferred embodiment, said stream D1 comprises a content by weight of chlorotrifluoroethylene of greater than 60%, advantageously greater than 70%, preferably greater than 80%, more preferentially greater than 85%, in particular greater than 90% on the basis of the total weight of said stream D1.

Preferably, step b2) is carried out by distillation at a pressure of less than 8 bara, preferably less than 6 bara. More preferentially, step b2) is carried out at a pressure of 1 to 6 bara.

Preferably, step b2) is carried out by distillation, and the temperature at the top of the distillation column is less than 40° C.; in particular, the temperature at the top of the distillation column is between −40° C. and 40° C.; more particularly, the temperature at the top of the distillation column is between −35° C. and 30° C.

Under these operating conditions, stream D1 is preferably in the form of an azeotropic or quasi-azeotropic composition comprising chlorotrifluoroethylene and 1,1,2-trifluoroethane.

Stream D1 as described in the present application is recycled to step a) of the present process. The steps of the present process are repeated. A fresh CTFE stream is mixed with stream D1 after recycling thereof to maintain the appropriate CTFE/H₂ ratio.

### Example

25 cm³ of catalyst (0.2% of palladium supported on α-alumina) are introduced into a tubular reactor consisting of a stainless steel tube with a length of 1200 mm over a diameter of 25 mm, and equipped with a jacket. The catalyst, thus charged, was subsequently activated in the following way: the reaction tube was placed in a tube furnace and was fed with a stream of hydrogen (from 0.05 to 0.1 mol per gram of catalyst). The catalytic bed is heated to a temperature of 200° C. to 250° C. with a temperature gradient of 0.2° C./min. After this activation period, the tube was cooled to ambient temperature and then was isolated in order to then be installed on a hydrogenolysis test bed.

4 test beds are used in parallel, each comprising a reactor prepared as described above. The four beds were fed with 1 mol/h of starting composition and 1 mol/h of hydrogen in anhydrous form. The temperature of the reacting jacket is 25° C. The contact time, calculated as being the ratio of the volume in liters of catalyst to the sum of the flow rates of the reactants in standard liters per second, was approximately 22 seconds. Tests are carried out using various starting compositions. Comparative example 1 was used starting with chlorotrifluoroethylene. Example 2 according to the invention was carried out starting from the chlorotrifluoroethylene used in the comparative example, to which 1,1,2-trifluoroethane (3.9%) was added.

The results are shown in table 1 below:

TABLE 1

| | H₂ flow | CTFE flow | Product |
|---|---|---|---|
| Examples | rate g/h | rate g/h | VF₃ g/h |
| Ex. 1 (Comp.) | 2 | 116.47 | 113.0 |
| Ex. 2 (invention) | 2 | 116.47 | 178.2 |

The productivity mentioned corresponds to the sum of the productivities obtained for all four hydrogenolysis beds. As can be seen, the trifluoroethylene productivity is significantly improved starting from the composition according to the invention compared with a chlorotrifluoroethylene composition without the additional compounds.

1-10. (canceled)

11. A process for producing trifluoroethylene in a reactor provided with a fixed catalytic bed comprising a catalyst, said process comprising the steps of:
a) reacting chlorotrifluoroethylene with hydrogen in the presence of the catalyst and in the gas phase in order to produce a stream A comprising trifluoroethylene;
b) treating said stream A under conditions sufficient to produce a stream D1 comprising 1,1,2-trifluoroethane in a content of less than 15% by weight on the basis of the total weight of said stream D1; and
c) recycling stream D1 to step a).

12. The process as claimed in claim 11, characterized in that stream A also comprises, in addition to trifluoroethylene, unreacted chlorotrifluoroethylene and 1,1,2-trifluoroethane.

13. The process as claimed in claim 11, characterized in that stream D1 also comprises chlorotrifluoroethylene in a content by weight of greater than 60% by weight on the basis of the total weight of said stream D1.

14. The process as claimed in claim 11, characterized in that stream D1 is in the form of an azeotropic or quasi-azeotropic composition comprising chlorotrifluoroethylene and 1,1,2-trifluoroethane.

15. The process as claimed in claim 11, characterized in that stream A comprises trifluoroethylene, chlorotrifluoroethylene and 1,1,2-trifluoroethane and step b) comprises the steps of:
b1) purifying said stream A comprising trifluoroethylene, chlorotrifluoroethylene and 1,1,2-trifluoroethane to form a stream C1 comprising trifluoroethylene and a stream C2 comprising chlorotrifluoroethylene and 1,1,2-trifluoroethane; and
b2) purifying stream C2 to produce said stream D1 and a stream D2 comprising 1,1,2-trifluoroethane.

16. The process as claimed in claim 15, characterized in that step b2) is carried out by distillation at a pressure of less than 8 bara.

17. The process as claimed in claim 15, characterized in that step b2) is carried out by distillation, and the temperature at the top of the distillation column is less than 40° C.

18. The process as claimed in claim 11, characterized in that said catalyst is a catalyst based on a metal from groups 8 to 10 of the Periodic Table of the Elements.

19. The process as claimed in claim 18, characterized in that the catalyst comprises palladium supported on α-alumina.

20. The process as claimed in claim 11, characterized in that the chlorotrifluoroethylene and the hydrogen are in anhydrous form.
